(19) United States
(12) Patent Application Publication
(10) Pub. No.: US 2004/0058895 A1
(43) Pub. Date: Mar. 25, 2004

(54) Multi-use vessels for vitamin D formulations

(75) Inventors: Richard B. Mazess, Madison, WI (US); Jeffrey W. Driscoll, Middleton, WI (US); Creighton Reed Goldensoph, DeForest, WI (US); Leon W. LeVan, Oregon, WI (US)

Correspondence Address: Michael Best & Friedrich LLP, One South Pinckney Street, P.O. Box 1806, Madison, WI, 53701-1806, US

(73) Assignee: Bone Care International, Inc., Middleton, WI

(21) Appl. No.: 10/608,480

(22) Filed: Jun. 27, 2003

Related U.S. Application Data
(63) Continuation-in-part of application No. 10/247,766, filed on Sep. 18, 2002.

(51) Int. Cl. A61K 31/59

(52) U.S. Cl. 514/167

(57) ABSTRACT

This invention relates to multi-use dispensing vessels containing pharmaceutical formulations of active vitamin D compounds.

### CROSS-REFERENCE TO RELATED APPLICATIONS

This application is a continuation-part application of U.S. patent application Ser. No. 10/247,766, filed Sep. 18, 2002.

### STATEMENT REGARDING FEDERALLY SPONSORED RESEARCH OR DEVELOPMENT

Not Applicable

This invention relates to multi-use dispensing vessels or vials containing pharmaceutical formulations of vitamin D compounds and analogs. The multi-use vials are particularly useful for parenteral administration. The invention also relates to plastic blow-fill containers containing pharmaceutical formulations of vitamin D compounds and analogs.

Drug formulations of, e.g., vitamin D compounds or analogs for intravenous administration are routinely supplied in single-use, single-dose glass ampoules or vials (glass vessels with a rubber top). For example, in end stage renal disease, a typical administration is that of a single container to a single hemodialysis patient at each dialysis session, often several times per week. The single-use container is standard for several reasons. Ampoules were the first containers used in an attempt to prevent contamination and oxidation of the active ingredients. The need to protect active compounds also led to the routine addition of antioxidants to preserve the integrity of the active agent. An example of a widely used antioxidant is ascorbic acid or sodium ascorbate, which is put into aqueous solutions of the active agent together with a surfactant to facilitate mixture. Drug formulations also have often been stabilized with buffers, such as acetate, citrate, glutamate, and phosphate buffers, to maintain pH, and chelating agents, such as citric acid, tartaric acid, amino acids, thioglycolic acid, and ethylenediamine tetraacetic acid disodium (EDTA). Subsequently, purely aqueous solutions have been replaced with solutions containing co-solvents, such as propylene glycol and an alcohol, in addition to water (See, U.S. Pat. No. 6,136,799 issued to Li et al). This combination has allowed certain drug compounds to be stablized in glass vials. Despite these various developments in single-use, single-dose ampoule or vial-contained formulations, there is an inherent high cost because the ampoules or vials are disposed of after the single use. Further, they remain costly to fabricate, are subject to breakage, and require a syringe and needle to load and inject the formulation.

Some cost savings are potentially available with multi-use vessels or vials. Multi-use vessels are suitable for multiple administrations in any desired partial amounts of a solution of an active substance, to either a single patient on multiple occasions or to multiple patients, or to some combination of different occasions and patients. In addition the larger volume of a multi-use vessel allows application of unusual dosage administrations (a large dose once-weekly versus smaller doses thrice-weekly). A multi-use vial makes dosing more efficient, minimizes wasted product, contains costs, and makes better use of storage space. In some clinic settings where multiple patients are treated with the same pharmaceutical formulation, a multi-use vial could provide significant savings, e.g., in reduced number and cost of containers.

However, special demands are made on the stability, sterility and storability of such forms of administration. Multi-use vials, which require a syringe and needle to be loaded with each dose, have a risk of contamination from reuse of the needle or syringe, or improper decontamination of the rubber membrane. Reports of infections transmitted through contaminated multidose vials clearly suggest that their use poses a tangible risk. Further, while common antimicrobial preservatives used in multidose vials may be highly effective for most bacteria, they are typically not antiviral agents. Also, there appears to be a vulnerable window of time (about two hours) during which contaminating organisms may remain viable in multi-use vials before the preservative fully exerts its effect. Even after the preservative inactivates the organism, endotoxins may be present and can cause pyretic or febrile reactions. While faulty aseptic technique is often the primary cause of vial contamination and significant microbial population, other factors include the design of the vial, storage conditions, the frequency of entering the vial, the environmental air injected into the vial and, of course, the nature of the drug formulation itself.

Needleless systems are now available for removal of the contents of multi-use vials, and can virtually eliminate the needle reuse, reentry risk. Because of multiple withdrawals from a vial, storage problems, etc., most existing formulations are neither suitable for nor approved for multi-use vials even with needleless systems.

New types of plastic containers (blow-fill) for intravenous drugs have also been developed which are of much lower cost and not subject to breakage. Moreover, these containers also can be made with a luer lock entry, thereby enabling them to be connected directly to a syringe or an indwelling catheter in a patient. This direct administration eliminates the need for a needle and syringe to withdraw and administer a dose. However, existing formulations e.g., for vitamin D analogs, do not allow these plastic containers to be used. Plastic containers are permeable to oxygen. A simple aqueous solution with an antioxidant is typically not stable, and the antioxidant, in some cases, is subject to degradation and/or discoloration. Also, a solution containing alcohol is likely to be unstable because the alcohol may interact chemically with the plastic.

The present invention provides multi-use vessels containing an active vitamin D formulation. The invention also provides needleless access vials and plastic blow-fill containers containing a vitamin D formulation suitable for human or veterinary administration on a multi-use basis and/or use in a high-dose episodic basis. A vitamin D formulation suitable for use in accordance with the present invention typically comprises 1) an active vitamin D compound or analog, 2) a non-ionic solubilizer, 3) a lipophilic antioxidant, and 4) optionally, an agent(s) that is an organic solvent, a preservative or both, in an aqueous vehicle. A suitable formulation for present the invention, thus, may comprise a therapeutically effective amount of a vitamin D compound or analog, a non-ionic solubilizer, a small amount of lipophilic antioxidant, and optionally, an agent that includes an organic solvent (e.g., ethanol) or co-solvents (e.g., propylene glycol and ethanol) and/or a preservative (e.g., benzyl alcohol).

Formulations suitable for use in accordance with the present invention may be formulated in a variety of concentrations in various vessel sizes for various administration dosages. The concentrations will depend on the particular vitamin D compound or analog used and the nature of the desired therapeutic response. For example, a multi-use vessel in accordance with the present invention may comprise a dose of doxercalciferol which is greater than 6 μg. Additionally, the dosage may be formulated in a vessel containing 4 mL or greater volume, or any other size vessel or container suitable for the formulation.

The invention also relates to methods for the treatment and prevention of certain diseases and disorders comprising employing a multi-use vessel, e.g., a needleless access vessel or a or plastic blow-fill vessel, containing a vitamin D formulation in accordance with the present invention to administer to a patient in need thereof an effective amount of such formulation. These diseases include hyperparathyroidism, especially secondary hyperparathyroidism, neoplastic diseases, such as cancers of the pancreas, breast, colon or prostate, other diseases of abnormal cell differentiation and/or cell proliferation such as leukemia, myelodysplastic syndrome, psoriasis, as well as disorders of calcium metabolism such as osteomalacia. In the treatment of hyperparathyroidism, especially secondary to chronic kidney disease, the multi-use vessel is employed to administer parenterally to a patient in need thereof an effective amount of the vitamin D composition contained in the vessel to lower elevated or maintain lowered blood (serum) parathyroid hormone level.

A fuller appreciation of the specific attributes of this invention will be gained upon an examination of the following more detailed description of the invention, and appended claims.

The present invention provides multi-use vessels, such as vials and plastic blowfill containers, utilizing a vitamin D formulation that provide single or multiple doses. A suitable pharmaceutical formulation typically comprises a therapeutically effective amount of an active vitamin D compound or analog, a non-ionic solubilizer, and a lipophilic antioxidant, and optionally, an agent that includes an organic solvent, a preservative or both, in an aqueous vehicle. An illustrated embodiment of the multi-use vessel includes a vessel volume greater than 2 mL and contains greater than 2 mL of the vitamin D formulation.

As used herein, the term “activated vitamin D” or “active vitamin D” in reference to a compound is intended to include any biologically active vitamin D compound, including a pro-drug (or pro-hormone), a precursor, a metabolite or an analog, in any stage of its metabolism. It is known that vitamin D compounds display a variety of biological activities, e.g., in calcium and phosphate metabolism (see, e.g., U.S. Pat. No. 5,104,864), as an antineoplastic agent (see, e.g., U.S. Pat. No. 5,763,429), and as an antihyperparathyroid agent (see, e.g., U.S. Pat. No. 5,602,116). It is contemplated that any of the biologically active forms of vitamin D can be used in the formulations in accordance with the present invention. Generally, an active vitamin D compound or analog is hydroxylated in at least the C-1, C-24 or C-25 position of the molecule, and either the compound itself or its metabolite binds to the vitamin D receptor (VDR).

Pro-drugs, for example, include vitamin D compounds that are, e.g., hydroxylated in the C-1. Such compounds undergo further hydroxylation in vivo, and their metabolites bind the VDR. Precursors include previtamins, such as 1α-hydroxyprevitamin D₂, 1α,24-dihydroxyprevitamin D₂, 1α,25-dihydroxyprevitamin D₂, 24-hydroxyprevitamin D₂, 1α,25-hydroxyprevitamin D₃ and 1α,25-dihydroxyprevitamin D₃, which are thermal isomeric forms of the vitamin forms. Metabolites generally include compounds or analogs that have undergone further metabolic processing, e.g., hydroxylation.

Examples of compounds suitable for formulations of the present invention include, without limitation, 1α,hydroxyvitamin D₂, 1α-hydroxyvitamin D₄, 1α,24-dihydroxyvitamin D₄, 1α,24-dihydroxyvitamin D₂, 1α,25-dihydroxyvitamin D₃ (calcitriol), 1α,25-dihydroxyvitamin D₂, 1α,25-dihydroxyvitamin D₄, 1α,24,25-dihydroxyvitamin D₂, 1α-hydroxyvitamin D₃ (alpha-calcidol), seocalcitol (EB-1089), calcipotriol, 22-oxacalcitriol (maxacalcitol), fluorinated compounds such as falecalcitriol, and 19-nor compounds such as paricalcitol. Among those compounds that have a chiral center, e.g., in the sidechain, such as at C-24, it is understood that both epimers (e.g., R and S) and the racemic mixture are within the scope of the present invention.

As used herein, the term “organic solvent” is also meant to include co-solvents, e.g., a combination of propylene glycol and ethanol.

As used herein, the term “preservative” is meant to refer to an antimicrobial agent, e.g., an anti-bacterial agent.

It also is understood that any numerical value recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between the lowest value and the highest value enumerated are to be considered to be expressly stated in this application.

The amount of selected vitamin D compound or analog is not critical to the present invention and may be varied to achieve the desired therapeutic response for a particular patient. The selected dosage also will depend on the activity of the specific compound or analog, the route of administration, the severity of the condition being treated, and the condition and history of the specific patient. For example, a therapeutic dose for active vitamin D compounds may range between about 2 μg and about 100 μg per dose.

Formulations suitable for use in the present invention may include a solubilizer or solubilizer agent. To some extent, the amount of active vitamin D in the formulations suitable for the present invention will be dependent on the solubility of the specific solubilizer used. Those skilled in the arts can adjust the ratios without undue experimentation. Suitable solubilizing agents for formulations of the present invention include non-ionic solubilizers. A non-ionic solubilizer is one where the hydrophilic part of the solubilizer carries no charge but derives its water solubility from highly polar groups such as hydroxyl or polyoxyethylene groups. Some surfactants known for use in the pharmaceutical field also have a solubilizing function.

Solubilizers generally include, without limitation, polyoxyalkylenes, cyclodextrins, dextrans, fatty acid esters of saccharose, fatty alcohol ethers of oligoglucosides (e.g., the akylpolyglucosides such as TRITON™), fatty acid esters of glycerol (as, e.g., glycerol mono/distearate or glycerol monolaurate), polyoxyethylene type compounds (PEGs, SOLUTOL™s, CREOMOPHOR™S, POE, PEO, Macrogol, Carbowax, Polyoxyl). The latter also include polyethoxylated fatty acid esters of sorbitan (e.g., polysorbates, such as TWEEN™s, SPAN™s), fatty acid esters of poly(ethylene oxide) (e.g., polyoxyethylene stearates), fatty alcohol ethers of poly(ethylene oxide) (e.g., polyoxyethylated lauryl ether), alkylphenol ethers of poty(ethylene oxide) (e.g., polyethoxylated octylphenol), polyoxyethylenepolyoxypropylene block copolymers (also known as poloxamers, such as “Pluronic”), and ethoxylated fats and oils (e.g., ethoxylated castor oil, or polyoxyethylated castor oil (also known as polyethylene glycol-glyceryl triricinoleate). Mixtures of solublilizers are also within the scope of the invention. Such mixtures are readily available from standard commercial sources. Solubilizers of particular interest include polysorbates, e.g., TWEEN™. The amount of such solubilizer present in the formulations of the present invention includes from about 0.05% to about 5% w/w.

A lipophilic antioxidant is also suitable in a typical formulation for use in accordance with the present invention. Suitable lipophilic antioxidants include, without limitation, butylated hydroxytoluene (BHT), lipoic acid, lycopene, lutein, lycophyll, xanthophyll, carotene, zeaxanthin or vitamin E and/or esters thereof. The lipophilic antioxidants are present in very small but effective amounts, e.g., about 20 to about 2000 ppm.

If desired, formulations suitable for use in accordance with the present invention can include optional agents. Examples of such agents include, without limitation, agents that are organic solvents, preservatives or both. Such agents include alcohols and polyols, such as ethanol, benzyl alcohol, isopropanol, butanol, ethylene glycol, propylene glycol, butanediols, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives. Amounts of optional agents include 0% to 30% w/w, e.g., organic solvent.

Multi-use vessels in accordance with the present invention may suitably include formulations of an active vitamin D compound, 0.05% to 5% w/w of a non-ionic solubilizer (e.g., a polysorbate), 20 to 2000 ppm lipophilic antioxidant (e.g., BHT), and optionally, 0% to 30% of an agent that is an organic solvent (or co-solvents), preservative, or both (e.g., benzyl alcohol).

A multi-use vessel for use in treating secondary hyperparathyroidism suitably includes a particular formulation that contains 6 to 100 μg 1α-hydroxyvitamin D₂ (doxercalciferol), 2.5% w/w benzyl alcohol, 0.5%-2.5% w/w TWEEN™-20, and 20 ppm BHT. Another formulation in accordance with the present invention suitable for treating secondary hyperparathyroidism includes 19-nor-vitamin D₂ or paricalcitol. Yet another suitable formulation includes 1α,25-dihydroxyvitamin D₃ (calcitriol).

A pharmaceutical formulation suitable for use in accordance with the present invention comprises an aqueous vehicle. The aqueous vehicle contains, of course, water, but it may also contain pH adjusting agents, stabilizing agents, solubilizing agent (see, hereinabove), isotonic adjusting agents, and solvents (e.g. organic solvents; as discussed above). Formulations in accordance with the present invention may suitably preclude the need for high organic solvent, which can cause irritation in some patients. In some cases, however, it may be appropriate to include an organic solvent or co-solvent, for example, in some cases up to 30% ethanol. The amount of water in a formulation in accordance with the present invention is normally at least about from about 50% to about 99% w/w.

For pharmaceutical formulations contained in the multi-use vessels of the present invention, the intended route of administration is suitably parenteral, i.e., for use by injection into, e.g., an animal or human body. Such route includes intravenous, intramuscular and subcutaneous administration, the intravenous route being especially suitable for the formulations of the present invention for use in connection with, e.g., secondary hyperparathyroidism or neoplastic disorders.

It is also understood that a formulation in accordance with the present invention may be contained in a vial, bottle, tube syringe or other container for multiple administrations. Such containers may be made of glass or a polymer material such as polypropylene, polyethylene, or polyvinylchloride. Containers may include a seal, or other closure system, such as a rubber stopper, or may be part of a needleless access system as described herein.

Multi-use dispensing vessels in accordance with the present invention contain formulations suitable for parenteral use, e.g., intravenous administration. Such vessels are commercially available and include the needleless access systems such as the InterLink™ I.V. Access System by Becton Dickinson, and SmartSite System by Alaris Medical Systems (see, also various needless transfer system in U.S. Pat. No. 6,045,538; U.S. Pat. No. 5,716,346; U.S. Pat. No. 6,406,455; U.S. Pat. No. 5,360,413; U.S. Pat. No. 5,549,577; WO 01/68017; WO 02/34198). Thus, it is understood that a formulation of the present invention is formulated for multi-use vessels, including in particular, those with needless access systems. Further, a suitable formulation is packaged with an insert from a governmental regulatory agency approving the multi-use of the vessel and formulation. In one embodiment, the vessels in accordance with the present invention are typically larger than 4 mL, a size that is larger than the size generally used for single dose formulations in single-use vials and ampoules. In another embodiment, the multi-vessel is a needless access vial.

Multi-use vessels containing formulations of the present invention can reduce the cost of providing such formulations. For example, a vitamin D formulation in a multi-use vessel in accordance with the present invention is suitable for use at a dialysis center, where, perhaps, 50 patients are dialyzed per day, and where such vessel can significantly reduce costs, i.e., one container versus 50 containers per day. Multi-use vessels containing a formulation in accordance with the present invention may be used in administering single doses to a patient or multiple patients on both a daily basis and/or an episodic basis. “Episodic dosing” is meant to refer to intermittent dosing, i.e., non-daily dosing, e.g., once per week, once every two weeks, twice a week, etc. Episodic dosing in accordance with the present invention may be suitably, e.g., once to three times per week, once every two weeks, once every three weeks or once per month.

Formulations in accordance with the present invention also are suitable for use in plastic (blow-fill) containers. For use in such containers, the blow-fill container formulations contain a solubilizer (e.g., a polysubstrate) a lipophilic antioxidant (e.g., BHT) and a preservative, e.g., an anti-microbial or anti-bacterial agent. For example, benzyl alcohol is a suitable preservative at a concentration of about 0.5% to about 3% w/w. Blow-fill container formulations include about 0.05% to about 5% w/w non-ionic solubilizer, about 20 to about 2000 ppm lipophilic antioxidant and about 0.5% to about 20% preservative. A typical blow-fill container formulation includes 0.05% to 5% w/w TWEEN™-20, 20 to 2000 ppm BHT, and 0.5% to 3% benzyl alcohol. Blow-fill containers can be made with a luer lock entry, thereby enabling them to be connected directly to a syringe or an indwelling catheter of a patient. Blow-fill containers containing formulations in accordance with the present invention may be used to administer doses to a single patient or multiple patients on a daily basis or an episodic basis, e.g., once to three times per week, once every two weeks, once every three weeks or once per month.

Formulations may be readily prepared by using pharmacopeia grade reagents in which the reagents are made up in stock solutions from which the resulting solutions at the appropriate concentrations can be made. Once the appropriate amounts of stock solution are combined, it is often desirable to stir the reagents for several minutes under nitrogen gas gently blown over the top of the mixture, i.e., a nitrogen gas overlay. Degassed water for injection is then added to the desired final volume, and stirring under nitrogen gas continued for another several minutes.

Additionally, as described hereinabove, vitamin D compounds in accordance with the present invention include prodrugs, i.e., drugs that require further metabolic processing in vivo, e.g., additional hydroxylation, as well as active vitamin D compounds that do not require further hydroxylation. Prodrugs of vitamin D compounds that have been found to be effective therapeutic agents are generally less reactive than the direct-acting compounds, e.g., the dihydroxy natural hormone, 1α,25-dihydroxyvitamin D₃.

In addition, formulations of the current invention may be terminally sterilized by means of, e.g., autoclaving.

A multi-use vessel in accordance with the present invention comprising a pharmaceutical formulation containing a vitamin D or a vitamin D analog like those substances described herein, is suitable for use in the treatment and/or prophylaxis of i) diseases or conditions characterized by abnormal cell differentiation and/or cell proliferation such as, e.g., psoriasis and other disturbances of keratinisation, neoplastic diseases and cancers, such as leukemia, myelodysplastic syndrome, pancreas, breast, colon and prostate cancers as well as skin cancer; ii) diseases of, or imbalance in, the immune system, such as host-versus-graft and graft-versus-host reaction and transplant rejection, and auto-immune diseases such as discoid and systemic lupus erythematosus, diabetes mellitus and chronic dermatoses of auto-immune type, e.g., scleroderma and pemphigus vulgaris; iii) inflammatory diseases such as rheumatoid arthritis, as well as in the treatment and/or prophylaxis of a number of (iv) other diseases or disease states, including hyperparathyroidism, particularly secondary hyperparathyroidism associated with chronic kidney disease, and in (v) promoting osteogenesis and treating/preventing bone loss as in osteoporosis and osteosmalacia. (For use of vitamin D compounds for treatment and prophylaxis of various diseases/disorders, see, e.g., U.S. Pat. Nos. 5,9722,917; 5,798,345; 5,763,428; 5,602,116; 5,869,386; 5,104,864; 5,403,831; 5,880,114; 5,561,123.) The vitamin D formulations in accordance with the present invention are especially suited for treatment of hyperparathyroidism, particularly secondary hyperparathyroidism associated with chronic kidney disease, i.e., for reducing elevated blood (serum) parathyroid hormone levels or maintaining reduced parathyroidism hormone levels, treatment of cell proliferative disorders such as psoriasis; disorders of the calcium metabolism, such as osteomalacia; or neoplastic diseases, such as cancers of the pancreas, breast, colon or prostate, where the method of treatment comprises administering to a patient in need thereof an effective amount of a vitamin D formulation in a dosage form which utilizes a multi-use vessel containing a formulation in accordance with the present invention. Daily dosages as well as episodic doses are contemplated.

Another aspect of the present invention relates to a pharmaceutical product that includes a composition of an active vitamin D compound in solution with a pharmaceutically or physiologically acceptable injectable carrier or excipient, a vessel for enclosing/containing the solution, and a notice in a form prescribed by a governmental agency that regulates the manufacture, use or sale of pharmaceuticals, which notice is reflective of approval by the agency of multi-use of the vessel for human or veterinary administration of the active vitamin D compound. Such notice, for example, may be labeling approved by the United States Food and Drug Administration for prescription drugs or an approved product insert. Compositions comprising an active vitamin D compound formulated in a compatible pharmaceutical carrier or excipient may be prepared, placed in an appropriate vessel, and labeled for treatment of an indicated condition and multi-use of the vessel. For example, the vessel suitably may have attached thereto a label indicating regulatory approval for the therapeutic indication and multi-use of the vessel. In a specific embodiment, the approval of use of the vessel is suitably for once per week administration or dosing. In another specific embodiment, the approval of use of the vessel is suitably for once every two week dosing.

In a specific embodiment, the vessel suitably has a content of vitamin D compound of at least 2.5 to 3 times the average conventional dose so that more than one patient may be treated from the vessel or so that a single-patient may be treated on multiple-occasions, or so that a single-patient may receive a high-dose, e.g., 2.5 to 3 times the conventional dose, on a once-weekly, bi-weekly, etc., basis. In another specific embodiment, the vessel is approved for administration of the higher dose of vitamin D compound in a once per week dosage. Less frequent episodic dosing is also contemplated. The vessel in accordance with the present invention is suitably large enough to contain a content of active vitamin D compound that is 2.5-3 times the typical dose, e.g., three times the standard container. For example, where the approved insert is for the treatment of hyperparathyroidism, the vessel suitably contains 3 μg or more of calcitriol, and is approved for once a week dosing. Alternatively, the vessel suitably contains 5 μg to 40 μg of calcitriol, and has regulatory approval for dosing in multiple patients on a basis of once, twice or thrice weekly. For example, the multi-use vessel may be used to administer five 1.1 μg doses of calcitriol given three times per week and three 3 μg doses of calcitriol given once weekly, or some other combination of once, twice or thrice weekly doses (including all dosing being once weekly in doses.) In other words, the vessel suitably has a volume greater than the standard container for vitamin D compounds and is approved by a regulatory agency for multi-use, and/or for use in a high-dose once-weekly administration. Other episodic dosing regimens are also contemplated.

As shown in Table 1 below, a once-weekly dose for treatment of secondary hyperparathyroidism is suitably 2.5-3 times higher than the average daily, twice weekly, or thrice weekly dose of active vitamin D compound typically administered at each dialysis session for a patient in renal failure. The largest single-use dose of the vitamin D agents available commercially is 1 μg for calcitriol and alphacalcidol, 10 μg for paricalcitol, and 4 μg for doxercalciferol.

TABLE 1

| Average | | Twice | ˜2.5 to 3 |
|---|---|---|---|
| dose per dialysis (μg) | | average dose | X dose average |
| Calcitriol | 1.1 | 2.2 | 3 |
| Paricalcitol | 5.0 | 10.0 | 13 |
| 22-oxa-calcitriol | 5.0 | 10.0 | 13 |
| doxercalciferol | 3.0 | 6.0 | 8 |
| alphacalcidol | 1.3 | 2.5 | 3 |

Without being bound by any particular theory, the high dose, reduced frequency regime is believed to provide efficacious treatment for elevated parathyroid hormone (PTH) blood levels because PTH levels are found to remain suppressed for a period of time after cessation of a therapeutic dose of active vitamin D. It is believed that these episodic, high dose regimes potentially reduce side effects such as hypercalcemia, especially for patients with elevated PTH (e.g., >400 pg/mL) who cannot be brought into the desired range of 150 to 300 pg/mL with the typical thrice-weekly dosing. In other words, a once-weekly, high dose of active vitamin D compound can maintain PTH levels despite the relatively short half-life of the active vitamin D. The vessel of this invention facilitates this mode of administration.

The present invention is further explained by the following examples which should not be construed by way of limiting the scope of the present invention.

### Preparation of Stock Solutions

### EXAMPLE 1

### Doxercalciferol (1α-hydroxyvitamin D₂) Stock Solution

12.558 mg of doxercalciferol was weighed and transferred to a 10-mL volumetric flask. The solid was diluted to volume with ethanol and the flask was vigorously shaken to dissolve the solid.

### EXAMPLE 2

### Butylated Hydroxytoluene (BHT) Stock Solution

2.22 g BHT was transferred to a 100-mL volumetric flask. The solid was diluted to volume with ethanol and the flask was vigorously shaken to dissolve the solid.

### EXAMPLE 3

### 10% Tween™-20

100 g Tween™-20KR was transferred to a 1-L volumetric flask and diluted to volume with degassed water for injection. A magnetic stir bar was added and the mixture stirred to mix.

### Formulations

### EXAMPLE 4

### Doxercalciferol Formulations

The general procedure for preparing doxercalciferol formulations was as follows. To a glass formulation vessel was added Doxercalciferol Stock Solution, 10% Tween™-20, BHT Stock Solution, and ethanol, in the order listed. Nitrogen gas was gently blown over the top of the mixture. A stir bar was added to the mixture and stirred for not less than 20 minutes while continuing the nitrogen gas overlay. Degassed water for injection was added to bring the final volume to one liter. The mixture was stirred for not less than 20 minutes while continuing the nitrogen gas overlay. The volumes of each component used in preparing the formulations are listed in the Table 1 below.

TABLE 1
Preparation of Doxercalciferol Formulations

| Doxercalciferol | Tween ™-20 | BHT Stock | Ethanol | Water for |
|---|---|---|---|---|
| Stock (mL) | Stock (mL) | (mL) | (mL) | Injection (mL) |
| 2.0 | 50 | 1.0 | 27 | 920 |
| 6.0 | 250 | 1.0 | 23 | 720 |

### EXAMPLE 5

### Multi-Use Multidose Vessel Formulation

A multi-dose formulation of calcitriol is prepared similarly to the methods described in Examples 1-4. Calcitriol is present at a content of greater than 3 μg in a volume greater than 2 mL.

A multidose formulation of doxercalciferol is prepared in a manner as described above in which doxercalciferol is present at a content of greater than 6 μg in a volume greater than 2 mL.

A multidose formulation of paricalcitol is prepared in a manner similar to the methods described above in Examples 1-4. Paricalcitol is present at a content of greater than 15 μg in a volume greater than 2 mL.

### EXAMPLE 6

### Blow-Fill Container Formulation

Doxercalciferol, BHT and TWEEN™-20 stock solutions are made similar to Examples 1-3. These stock solutions are used to prepare specific solutions suitable for blow-fill containers similar to Example 4, except benzyl alcohol is used to prepare the formulation and has final concentrations of 0.5% to 3% w/w.

In summary, the present invention provides a multi-use vessel containing vitamin D compounds. The multi-use dispensing vessels may be suitably a needleless access vials or plastic (blow-fill) containers.

All patents, publications and references cited herein are hereby fully incorporated by reference. In case of conflict between the present disclosure and incorporated patents, publications and references, the present disclosure should control.

While the present invention has now been described and exemplified with some specificity, those skilled in the art will appreciate the various modifications, including variations, additions, and omissions, that may be made in what has been described. Accordingly, it is intended that these modifications also be encompassed by the present invention and that the scope of the present invention be limited solely by the broadest interpretation that lawfully can be accorded the appended claims.

What is claimed is:

1. A pharmaceutical product comprising:
a composition comprising an active vitamin D compound in a physiologically acceptable injectable form;
a vessel containing the vitamin D compound; and
a notice in a form prescribed by a governmental agency regulating the manufacture, use or sale of pharmaceuticals, which notice reflects approval by the agency for multi-use of the vessel for dispensing the active vitamin D compound for human or veterinary administration.

2. The product of claim 1, wherein use of the vessel is approved for episodic dosing for the active vitamin D Compound.

3. The product of claim 2, wherein the episodic dosing is once-weekly dosing of the active vitamin D compound.

4. The product of claim 1, wherein the vitamin D compound is selected from the group consisting of calcitriol, paricalcitol, doxercalciferol, alphacalcidol and 22-oxa-calcitriol.

5. The product of claim 2, wherein the active vitamin D compound is calcitriol and the vessel contains 3 μg or more of calcitriol.

6. The product of claim 3, wherein the once-weekly dosing is about 3 μg of calcitriol.

7. The product of claim 2, wherein the active vitamin D compound is paricalcitol and the vessel contains 13 μg or more of paricalcitol.

8. The product of claim 3, wherein the once-weekly dosing is about 13 μg of paricalcitol.

9. The product of claim 2, wherein the active vitamin D compound is doxercalciferol and the vessel contains 8 μg or more of doxercalciferol.

10. The product of claim 3, wherein the once-weekly dosing is about 8 μg of doxercalciferol.

11. The product of claim 2, wherein the active vitamin D compound is alphacalcidol and the vessel contains 3 μg or more of alphacalcidol.

12. The product of claim 3, wherein the once-weekly dosing is about 3 μg of alphacalcidol.

13. The product of claim 2, wherein the active vitamin D compound is 22-oxacalcitriol and the vessel contain 13 μg or more of 22-oxa-calcitriol.

14. The product of claim 3, wherein the once-weekly dosing is about 13 μg of 22-oxa-calcitriol.

15. The product of claim 2, wherein the vessel is approved for bi-weekly dosing of the active vitamin D compound.

16. The product of claim 1, wherein the vessel is a needleless access vial.

17. The product of claim 2, wherein the vessel is a needleless access vial.

18. The product of claim 3, wherein the vessel is a needleless access vial.

19. The product of claim 1, wherein the use of the vessel is further approved for high-dose, once-weekly administration.

20. The product of claim 2, wherein the use of the vessel is further approved for high-dose, once-weekly administration.

21. A vessel, comprising:
a composition comprising an active vitamin D compound in solution in a physiologically acceptable injectable carrier contained therein; and
a notice associated with the vessel in a form prescribed by a governmental agency regulating the manufacture, use or sale of pharmaceuticals, which notice reflects approval by the agency for multi-use of the vessel for dispensing the active vitamin D compound for treatment of an indicated condition.

22. The vessel of claim 21, wherein the indicated condition is selected from the group consisting of hyperparathyroidism, secondary hyperparathyroidism, cancers of the pancreas, breast, colon or prostate, leukemia, myelodysplastic syndrome, psoriasis, and calcium metabolism disorders.

23. The vessel of claim 21, wherein the use of the vessel is approved for once-weekly administration of the active vitamin D compound.

24. The vessel of claim 21, wherein the vitamin D is selected from the group consisting of calcitriol, paricalcitol, doxercalciferol, alphacalcidol, and 22-oxa-calcitriol.

25. The vessel of claim 21, wherein the vessel holds a volume that is greater than 4 mL.

26. The vessel of claim 21, wherein the vessel contains 3 μg or more of calcitriol.

27. The vessel of claim 21, wherein the use of the vessel is further approved for high-dose, once-weekly administration.

28. The vessel of claim 21, wherein the indicated condition is secondary hyperparathyroidism.

29. The vessel of claim 28, wherein the active vitamin D compound is present in a content sufficiently large to permit once-weekly doses that are 2.5 to 3 times the average conventional dose given for secondary hyperparathyroidism.

30. The vessel of claim 21, wherein the vessel is a plastic blow-fill container.

31. The vessel of claim 21, wherein the vessel is a needleless access vial.

32. A pharmaceutical product, comprising:
a multi-use, needleless-access vessel or plastic blow-fill container,
a composition comprising an active vitamin D compound in injectable form contained in the vessel; and
a notice associated with the vessel in a form prescribed by a governmental regulatory agency regulating the manufacture, use or sale of pharmaceutics, which notice is reflective of approval by the agency of the vitamin D compound for human or veterinary administration to treat hyperparathyroidism.

33. The pharmaceutical product of claim 32, wherein the hyperparathyroidism is secondary hyperparathyroidism.

34. A pharmaceutical product, comprising: a multi-use, needleless-access vessel or a plastic blow-fill container; a composition comprising an active vitamin D compound in injectable form contained in the vessel; and a notice associated with the vessel in a form prescribed by a governmental regulatory agency regulating the manufacture, use or sale of pharmaceutics, which notice is reflective of approval by the agency of the vitamin D compound for human or veterinary administration to treat hyperparathyroidism; the approval being for once-weekly dose administration; the dose being at least 2.5 to 3 times the average conventional dose for treatment of hyperparathyroidism.

35. A method of treating a hyperproliferative diseases comprising utilizing the vessel of claim 21 to parenterally administer to a mammal in need thereof an effective amount of the composition contained in the vessel.

36. A method of treating hyperparathyroidism comprising utilizing the vessel of claim 21 to administer to a mammal in need thereof an effective amount of the composition contained in the vessel.

37. The method of claim 36 wherein the dose is administered once-weekly.

38. The method of claim 36 wherein the dose is administered bi-weekly.

39. The method of claim 36 wherein the active vitamin D is present in amount that is 2.5 to 3 times the average conventional dose given for hyperparathyroidism.

40. The method of claim 36 wherein the vessel is a needleless access vial.

41. The method of claim 36 wherein the vessel is a plastic blow-fill container.

42. The method of claim 36, wherein the hyperparathyroidism is secondary to chronic kidney disease.

43. A method of treating calcium metabolism disorders comprising utilizing the vessel of claim 21 to parenterally administer to a mammal in need thereof an effective amount of the composition contain in the vessel.

44. A method of lowering elevated or maintaining lowered blood parathyroid hormone level in patients suffering from hyperparathyroidism secondary to chronic kidney disease, comprising utilizing the vessel of claim 21 to parenterally administer to the patients an effective amount of the composition contained in the vessel.

45. The method of claim 44, wherein the composition is administered once-weekly.

46. The method of claim 44, wherein the composition is administered in high dose.

47. The method of claim 46, wherein the high dose is 2.5 to 3 times the average conventional dose given for hyperparathyroidism.

48. The method of claim 46, wherein the high dose is 8 μg or more of doxercalciferol.

49. The method of claim 46, wherein the high dose is 3 μg or more of calcitriol.

50. The method of claim 46, wherein the high dose is 13 μg or more of paricalcitol.
